(19) United States
(12) Patent Application Publication
(10) Pub. No.: US 2026/0076762 A1
(43) Pub. Date: Mar. 19, 2026

(54) METHOD FOR ROBOTIC ASSISTED HEART TRANSPLANT

(71) Applicant: KING FAISAL SPECIALIST HOSPITAL & RESEARCH CENTRE, Riyadh (SA)

(72) Inventor: Feras KHALIEL, Riyadh (SA)

(73) Assignee: KING FAISAL SPECIALIST HOSPITAL & RESEARCH CENTRE, Riyadh (SA)

(21) Appl. No.: 19/322,919

(22) Filed: Sep. 9, 2025

Related U.S. Application Data
(60) Provisional application No. 63/694,512, filed on Sep. 13, 2024.

(51) Int. Cl. A61B 34/00; A61B 17/02; A61B 17/11; A61K 38/17; A61L 31/04; A61M 1/36

(52) U.S. Cl. CPC A61B 34/70; A61B 17/0218; A61B 17/11; A61K 38/1709; A61L 31/048; A61M 1/3659; A61M 1/3666; A61B 2017/0237; A61B 2017/1107; A61L 2430/20

(57) ABSTRACT

A method for performing a robotic-assisted orthotopic heart transplant includes forming a working port subareolarly by a mini-thoracotomy at a working intercostal space, forming additional ports for robotic arms, and arranging robotic arms through the ports. A first robotic arm with first forceps extends through a second port lateral to a mid-clavicular line, while second and third robotic arms containing an imaging device and second forceps operate through the working port. A fourth robotic arm with scissors or needle holder extends through a third port proximal to an anterior axillary line. The method includes cannulating for cardiopulmonary bypass, opening the pericardium, cross-clamping the aorta, removing the native heart, and performing sequential anastomoses of the donor heart including the left atrium, pulmonary artery, aorta, inferior vena cava, and superior vena cava.

### CROSS-REFERENCE TO RELATED APPLICATION

The present application claims benefit of priority to U.S. Provisional Application No. 63/694,512 having a filing date of Sep. 13, 2024, and which is incorporated herein by reference in its entirety.

### BACKGROUND

### Technical Field

The present disclosure is generally directed to the field of cardiac surgery and transplantation. More specifically, the present disclosure pertains to robotic-assisted surgical techniques for performing orthotopic heart transplantation procedures.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Heart failure represents a major global health burden affecting millions of patients worldwide, with end-stage heart disease being particularly devastating due to its poor prognosis and impact on quality of life. The prevalence of heart failure in Western countries is estimated between 1-2% of the general population, with annual incidence rates approaching 5-10 cases per 1000 persons. Current statistics indicate that the 30-day mortality rate for patients with heart failure remains alarmingly high at 10-20% [See: Mosterd, A.; Hoes, A. W. Clinical epidemiology of Heart Failure. Heart.2007;93(9):1137-1146], highlighting the critical nature of this condition. The growing prevalence of heart failure, coupled with an aging population and increasing rates of cardiovascular risk factors, has led to a substantial rise in the number of patients requiring heart transplantation as a definitive treatment option. Traditional treatment approaches have faced significant challenges in addressing this increasing demand while maintaining optimal patient outcomes.

Conventional heart transplantation procedures have historically been performed through a full median sternotomy approach, which involves a substantial vertical incision through the sternum to provide direct access to the heart and great vessels. This traditional open surgical technique, while providing good exposure and access to cardiac structures, is associated with significant postoperative pain, prolonged recovery periods, and extended hospital stays lasting several weeks. The invasive nature of the median sternotomy approach also carries risks of sternal wound complications, including infection and delayed healing, which can significantly impact patient recovery and quality of life. Additionally, the extensive tissue trauma associated with this approach often results in increased blood loss, greater transfusion requirements, and heightened inflammatory responses that can complicate the immediate post-transplant period.

Recent advances in minimally invasive cardiac surgery have demonstrated numerous benefits, including reduced postoperative pain, shortened hospital stays, faster return to normal activities and improved cosmesis [See: Mod, i P.; Hassan, A. Chitwood, W. R. Jr. Minimally invasive mitral valve surgery: a systematic review and meta-analysis. European Journal of Cardio-Thoracic Surgery. 2008;34(5):943-952]. These techniques have been successfully applied to various cardiac procedures, including valve repairs and replacements, coronary artery bypass grafting, and the treatment of congenital heart defects. Robotic surgical systems, in particular, have emerged as powerful tools for enhancing surgical precision and enabling complex cardiac procedures to be performed through smaller incisions. The integration of robotic assistance has shown promising results in terms of improved visualization, enhanced dexterity, and reduced surgical trauma across multiple cardiac surgical applications.

Robotic transplantation is being successfully used for kidney transplant [See: Breda, A.; Territo, A.; Gausa, L.; et al. Robot-assisted kidney transplantation: the European experience. European Urology. 2018;73(2):273-281], lung transplant [See: Emerson, D.; Catarino, P.; Rampolla, R.; et al. Robotic-assisted lung transplantation: First in man. The Journal of Heart and Lung Transplantation.2024; 43(1):158-161] and liver transplant [See: Khan, A. S., Scherer, M., Panni, R., et al. Total robotic liver transplant: the final frontier of minimally invasive surgery. American Journal of Transplantation. 2024;24(8):1467-1472]. Surgical robots have been developed to enhance surgical ability and precision, and to repair structural heart conditions, including mitral valve repair (MV repair), atrial septal defect closure, cardiac tumor resection. The most common applications in cardiac surgery are MV repair and endoscopic coronary artery bypass grafting (CABG) [See: Ishikawa, N.; Watanaba, G. Robot-Assisted Cardiac Surgery. Annals of Thoracic and Cardiovascular Surgery. 2015; 4:322-328].

However, despite these advances in minimally invasive cardiac surgery, several technical challenges have historically limited the application of robotic assistance in heart transplantation procedures. These limitations have included concerns about adequate exposure and access for performing complex vascular anastomoses, managing donor heart preservation and implantation through smaller incisions, and maintaining optimal surgical precision during critical phases of the procedure. The technical complexity of heart transplantation, combined with the time-sensitive nature of donor organ preservation, has posed significant challenges to the development of minimally invasive approaches.

EP3042625B1 describes a cooperative minimally invasive telesurgical system with multiple robotic arms controlled through master controls, including techniques for performing cardiac procedures through apertures along the right side of a patient. The proposed system enables control of multiple manipulator arms through a single console while providing image capture and display capabilities for visualizing the surgical site. The reference discusses positioning of entry ports and establishment of pivot points for the robotic arms relative to anatomical landmarks. However, this reference does not describe integration of specific sequencing of robotic arm arrangements through precisely positioned ports, coordinated cannulation techniques, and systematic anastomotic steps performed through a working port while maintaining cardiopulmonary bypass, enabling completion of a full orthotopic heart transplant procedure through a minimally invasive approach.

US20200323593A1 describes methods and apparatus for surgical planning in robotic surgery systems, particularly focusing on optimal placement of entry ports and robot positioning for minimally invasive procedures. The system processes imaging data to create surgical site models and determines advantageous entry port locations based on multiple criteria including robot constraints, anatomical constraints, and surgeon preferences. The planning system includes validation and simulation capabilities to ensure feasibility of selected port placements and robot positions. However, this reference does not describe integration of specific sequencing of robotic arm arrangements through precisely positioned ports, coordinated cannulation techniques, and systematic anastomotic steps performed through a working port while maintaining cardiopulmonary bypass, enabling completion of a full orthotopic heart transplant procedure through a minimally invasive approach.

The CTSNet publication describes implementation of robotic cardiac surgery techniques at specialized centers, detailing approaches for port placement and surgical access through mini-thoracotomy incisions. This reference outlines considerations for patient positioning, instrument selection, and surgical workflow in robotic cardiac procedures performed through right-sided approaches. However, this reference does not describe integration of specific sequencing of robotic arm arrangements through precisely positioned ports, coordinated cannulation techniques, and systematic anastomotic steps performed through a working port while maintaining cardiopulmonary bypass, enabling completion of a full orthotopic heart transplant procedure through a minimally invasive approach.

Each of the aforementioned references suffers from one or more drawbacks hindering their adoption, such as limited integration between robotic assistance and complex cardiac procedures, challenges in coordinating multiple surgical steps through minimally invasive approaches, difficulties in maintaining optimal surgical access while performing intricate vascular anastomoses, and complications in managing cardiopulmonary support during minimally invasive cardiac operations. Accordingly, it is one object of the present disclosure to provide a method for performing a robotic-assisted orthotopic heart transplant that addresses these limitations while maintaining surgical efficacy and patient safety through an integrated approach to minimally invasive cardiac transplantation. The present method integrates specific sequencing of robotic arm arrangements through precisely positioned ports, coordinated cannulation techniques, and systematic anastomotic steps performed through a working port while maintaining cardiopulmonary bypass, enabling completion of a full orthotopic heart transplant procedure through a minimally invasive approach.

### SUMMARY

In an exemplary embodiment, a method for performing a robotic-assisted orthotopic heart transplant is described, comprising: forming a working port subareolarly by a mini-thoracotomy at a working intercostal space of a patient, the working port being the first port; forming a second port and a third port configured to receive robotic arms therein; arranging a first robotic arm through the second port disposed at a second intercostal space lateral to a mid-clavicular line, the first robotic arm including first forceps; arranging a second robotic arm and a third robotic arm in the working port, the second robotic arm including an imaging device and the third robotic arm including second forceps; arranging a fourth robotic arm through the third port disposed at a fourth intercostal space proximal to an anterior axillary line, the fourth robotic arm including scissors or a needle holder; cannulating the patient in a right groin for cardiopulmonary bypass with an arterial cannula and a multistage femoral venous cannula for inferior vena cava (IVC) drainage, and inserting a cannula percutaneously into a right internal jugular vein for superior vena cava (SVC) drainage; initiating cardiopulmonary bypass upon achieving a predetermined activated clotting time (ACT), opening a pericardium anteriorly and retracting the pericardium laterally, and dissecting around the SVC, an IVC, an aorta, and a pulmonary artery; cross-clamping a distal ascending aorta with a cross-clamp, snaring the SVC and IVC, excising a native heart, and removing the native heart via the working port; arranging, through the working port, a donor heart in the patient anatomically; performing anastomosis of a left atrium using a first suture; performing anastomosis of the pulmonary artery using a second suture; performing anastomosis of the aorta using a third suture and inserting a root vent connected to low suction with cardiopulmonary bypass; performing anastomosis of the IVC using a fourth suture and removing the aortic cross-clamp; and performing anastomosis of the SVC using a fifth suture on the donor heart.

In some embodiments, the method further comprises arranging a patient in a left lateral decubitus position under general anesthesia with a double lumen endotracheal tube.

In some embodiments, the method further comprises inserting a central venous line and a pulmonary artery catheter through a left internal jugular vein.

In some embodiments, the pulmonary artery catheter is a Swan Gans catheter.

In some embodiments, the method further comprises arranging a multiport robotic arm platform proximal to the right side of the patient, the multiport robotic arm platform configured to control the robotic arms.

In some embodiments, the method further comprises filling the donor heart to check all suture lines, gradually weaning off the cardiopulmonary bypass machine on minimal inotropic support, decannulating the patient, and administering protamine.

In some embodiments, the method further comprises upon determining a leak from suture, placing additional stitches to stop the leak.

In some embodiments, the method further comprises performing postoperative transesophageal echocardiography to confirm good left ventricular function and mildly reduced right ventricular function.

In some embodiments, the working port has a diameter of 4 to 6 cm.

In some embodiments, forming the working port further comprises making an incision having a length of 6 to 10 cm.

In some embodiments, the second port 7 to 9 mm.

In some embodiments, the third port has a diameter of 7 to 9 mm.

In some embodiments, the method further comprises applying a sterile gel to the working port before arranging the donor heart through the working port.

In some embodiments, the initiating pulmonary bypass further comprises administering heparin.

In some embodiments, the performing the anastomosis of the IVC and removing the aortic cross-clamp further comprises removing the aortic cross-clamp while raising an aortic root vent suction to 500 cc/min.

In some embodiments, the cannulating the patient in the right groin for cardiopulmonary bypass further comprises cannulating the patient in the right groin for cardiopulmonary bypass under echocardiography guidance.

In some embodiments, the cannulating the patient in the right groin for cardiopulmonary bypass uses an open groin technique.

In some embodiments, the method further comprises performing a computed tomography (CT) scan of the patient to determine an anatomy of the patient.

In some embodiments, the method further comprises determining locations of the working port, the second port, and the third port based on the determined anatomy of the patient via the CT scan.

In some embodiments, the ACT is 480 s or greater.

In some embodiments, a material of the first suture is 3/0 polypropylene.

In some embodiments, a material of the second, third and fourth suture is CV-4 Gore-Tex.

In some embodiments, a material of the fifth suture is 5/0 propylene.

In some embodiments, in preparation for robotic heart transplant, a suitable donor is selected and matched for blood group, human leukocyte antigen (HLA) and body weight.

In some embodiments, the recipient's preoperative computerized tomography (CT) scan for chest, abdomen and pelvis was obtained to delineate suitable intrathoracic anatomy and peripheral vasculature for the suitability of cardiopulmonary bypass cannulation.

In some embodiments, under general anesthesia with a double lumen endotracheal tube, a central venous line and Swan Ganz catheter were inserted through left internal jugular vein.

In some embodiments, the recipient or patient was positioned in a slight left lateral decubitus position.

In some embodiments, after sterile prepping and draping, a jugular femoral cardiopulmonary bypass (CPB) cannulation setup was performed after giving Heparin, i.e., a 19Fr cannula was inserted percutaneously in a right internal Jugular vein for superior vena cava (SVC) drainage, and a 17Fr arterial cannula was inserted into a right femoral artery by open technique and a 25Fr multistage venous cannula for inferior vena cava (IVC) drainage.

In some embodiments, a sub-areolar 6-7 cm thoracotomy was performed (at a working port) in a 4^{th} intercostal space.

In some embodiments, after achieving target activating clotting time (ACT), CPB was initiated. After achieving full flows, ventilation was disconnected.

In some embodiments, a Da Vinci Xi® surgical system was docked and ports were arranged in the following manner: the port for robotic arm no. 1 was inserted at a 2nd intercostal space lateral to mid clavicular line, the ports for robotic arms no. 2 and 3 were inserted directly through the working port, and the port for robotic arm no. 4 was inserted through a 6th intercostal space anterior axillary line. DeBakey forceps were used at arm no. 1, a 30-degree camera at arm no. 2, Cadiere forceps at arm no. 3 and scissors/needle holders (alternatively) were used at arm no. 4.

In some embodiments, the pericardium was opened anteriorly, with the help for retraction sutures, retracted laterally.

In some embodiments, the SVC and IVC were mobilized and snared.

In some embodiments, space between aorta and pulmonary artery was created.

In some embodiments, a Chitwood cross clamp was applied.

In some embodiments, the native heart was excised and extracted via the working port.

In some embodiments, the donor heart was prepared by applying identification marks at left atrium (LA) close to the left atrial appendage, anterior surfaces of the pulmonary artery (PA) and the SVC.

In some embodiments, the donor heart was inserted through working port smoothly and positioned anatomically.

In some embodiments, with the help of the Cadiere forceps, the heart was positioned in anatomically to perform anastomosis. The needle holder and DeBakey forceps were utilized to perform anastomosis.

In some embodiments, the first anastomosis was of LA performed using 3/0 polypropylene continuous stitch, followed by end to end anastomosis of the PA, aorta and IVC performed using a Gore-Tex CV-4® suture.

In some embodiments, an aortic root vent was inserted and connected to suction for deairing, and the cross clamp was released. The right ventricular and right atrial pacing wires were placed and the heart was paced.

In some embodiments, the last anastomosis of SVC was performed using 5/0 polypropylene.

In some embodiments, the heart was given sufficient time for re-perfusion before coming off CPB.

In some embodiments, additional 4/0 polypropylene pledgeted stitches were applied for potential leak points before coming off CPB.

In some embodiments, after coming off CPB, a trans esophageal echo cardiography was performed and revealed good left ventricular function.

In some embodiments, the patient was decannulated from CPB and protamine was administered.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings.

FIG. 1 is a flowchart illustrating steps of a method for performing a robotic-assisted orthotopic heart transplant, according to certain embodiments.

FIG. 2 is a schematic illustration showing patient positioning and port placement for the robotic-assisted orthotopic heart transplant procedure, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of this disclosure are directed to a method for performing a robotic-assisted orthotopic heart transplant that combines surgical robotics, minimally invasive access techniques, and surgical planning to enable complete cardiac transplantation through optimized port placement and instrument coordination. The method integrates multiple surgical phases including patient positioning, strategic port placement, establishment of cardiopulmonary support, precise robotic instrument manipulation, and systematic completion of critical surgical steps through carefully planned minimally invasive approaches. This integrated approach enables surgeons to perform complex cardiac transplantation procedures while potentially reducing surgical trauma and optimizing procedural workflow.

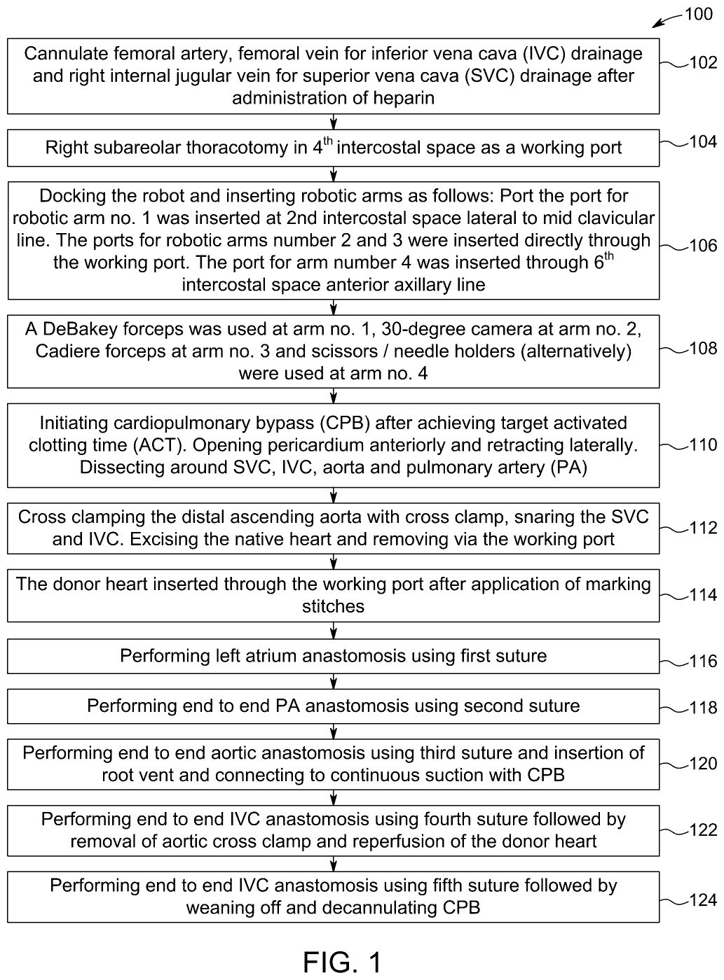

Referring to FIG. 1, illustrated is a flowchart of a method (as represented hereinafter by reference numeral 100) for performing a robotic-assisted orthotopic heart transplant, as per embodiments of the present disclosure. The method 100 provides a comprehensive surgical approach that integrates multiple technological and procedural steps into a cohesive system for minimally invasive cardiac transplantation. The method 100 enables adaptation to different surgical settings and team configurations while preserving procedural standardization. This comprehensive approach addresses multiple aspects of surgical workflow, including space management, instrument coordination, and team communication, while maintaining focus on surgical precision and patient safety. The method 100 emphasizes reproducibility and standardization while allowing for necessary adaptations to individual patient needs and anatomical variations. The method 100 creates a structured environment for performing complex cardiac procedures while potentially reducing technical barriers to minimally invasive cardiac transplantation.

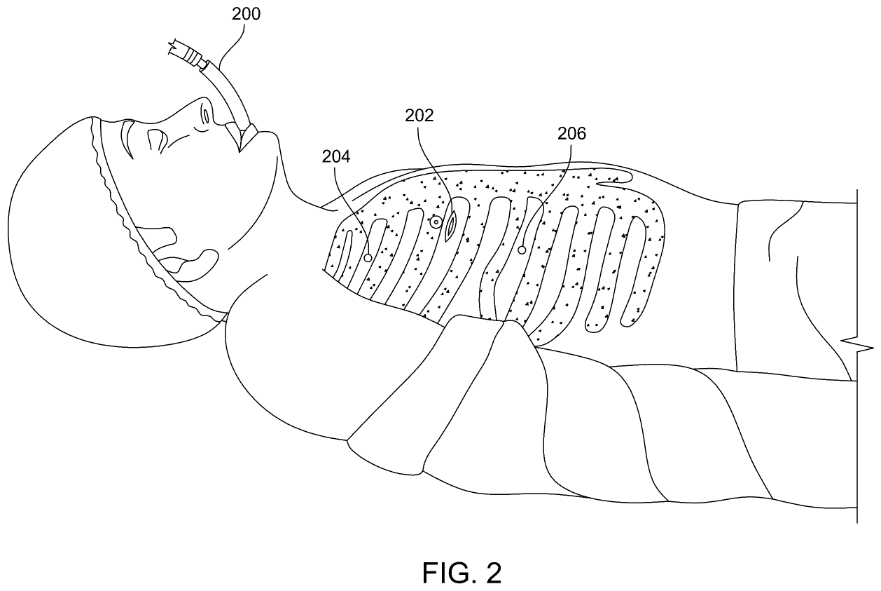

Preceding the main surgical procedure, the method 100 includes arranging a patient in a left lateral decubitus position under general anesthesia with a double lumen endotracheal tube 200 (as shown in FIG. 2). The left lateral decubitus positioning enables access to the right thoracic cavity. The double lumen endotracheal tube 200 allows single lung ventilation during the procedure. Herein, the anesthesia administration follows protocols for cardiac procedures. It may be understood that the patient positioning incorporates support structures to maintain stable alignment throughout the procedure.

The method 100 also includes inserting a central venous line and a pulmonary artery catheter through a left internal jugular vein. The central venous line placement enables continuous monitoring of central venous pressure. The catheter positioning enables monitoring of hemodynamic parameters throughout the procedure. Herein, the left internal jugular vein approach prevents interference with the right-sided surgical field. The insertion process follows standard sterile techniques for central line placement.

In an embodiment, the pulmonary artery catheter is a Swan Gans catheter. That is, the method 100 utilizes the Swan Gans catheter as the pulmonary artery catheter. The Swan Gans catheter enables measurement of pulmonary artery pressures. The catheter provides continuous monitoring of cardiac filling pressures. The proposed positioning may follow specific protocols for proper placement within the pulmonary circulation.

The method 100 further includes arranging a multiport robotic arm platform (not shown) proximal to a left side of the patient, the multiport robotic arm platform configured to control the robotic arms (not shown). The multiport robotic arm platform setup includes specific configurations for cardiac procedures. The proposed positioning of the multiport robotic arm platform enables optimal reach for all robotic arms. The arrangement is maintained to ensure proper clearance for surgical team movement.

Referring now to the flowchart of FIG. 1 and the schematic of FIG. 2 in combination, the details about main surgical procedure are discussed in detail. Herein, at step 102, the method 100 includes cannulating the patient in a right groin for cardiopulmonary bypass with an arterial cannula and a multistage femoral venous cannula and inserting a cannula percutaneously into a right internal jugular vein for superior vena cava (SVC) drainage. The arterial cannula establishes arterial flow for the cardiopulmonary bypass circuit. The multistage femoral venous cannula provides venous drainage from multiple levels within the inferior vena cava. The percutaneous right internal jugular vein cannula ensures complete venous drainage through the SVC. The cannulation process follows a specific sequence to establish complete cardiopulmonary support while maintaining hemodynamic stability.

In some embodiments, the cannulating the patient in the right groin for cardiopulmonary bypass includes performing the cannulation under echocardiography guidance. Herein, the echocardiography visualization-based guidance prevents potential complications during the cannulation process. Specifically, the echocardiography guidance enables real-time visualization of cannula positioning within the vascular structures. Further, the echocardiographic guidance confirms proper placement of the multistage femoral venous cannula within the inferior vena cava. Such guidance system ensures optimal positioning of the arterial cannula within the femoral artery.

In some embodiments, the cannulating the patient in the right groin for cardiopulmonary bypass uses an open groin technique. The open groin technique involves surgical exposure of the femoral vessels prior to cannulation. The open groin technique allows secure placement of purse-string sutures for cannula stabilization. The proposed process enables direct visualization of the femoral artery and vein during cannula insertion. Such direct access, in turn, facilitates safe decannulation at the conclusion of the procedure.

At step 104, the method 100 includes forming a working port subareolarly by a mini-thoracotomy at a 4^{th} intercostal space of a patient (which can herein also be referred to as a “first port”), the first port being a working port 202. As depicted in FIG. 2, the working port 202 is positioned to provide access for both robotic instruments and direct visualization for the transplant procedure. The subareolar positioning is chosen to optimize the relationship between the port and the underlying cardiac structures while providing consistent anatomical landmarks across different patient populations. The working port 202 serves as the primary access point for both the removal of the native heart and subsequent insertion of the donor heart, therefore requiring consideration of its size and position relative to chest wall anatomy of the patient.

The working port 202 is defined to facilitate the introduction of surgical instruments and provides sufficient space for manipulation during the various phases of the transplant procedure. The formation of the working port 202 involves surgical dissection through multiple tissue layers while maintaining careful hemostasis. For present purposes, the intercostal space selected for the working port 202 is evaluated preoperatively to ensure adequate spacing between ribs and optimal alignment with the intended surgical target area. The position of the working port 202 is planned to align with anatomical landmarks of the patient and also to optimize the surgical approach through the intercostal space. The position of the working port 202 can be further selected to minimize interference with the surrounding tissues while maintaining adequate access to the surgical field.

In an embodiment, the working port 202 has a diameter of, for example, 3 to 8 cm, or 3.5 to 7 cm, or 4 to 6 cm. The given diameter range is selected to provide adequate access for surgical instruments and organ manipulation while maintaining the minimally invasive nature of the procedure. Such diameter range for the working port 202 allows for precise control of surgical instruments while minimizing tissue trauma. The diameter is also selected to accommodate the size requirements for donor heart insertion and native heart removal. The specified diameter range enables required instrument manipulation and optimal visualization throughout the procedure.

In some embodiments, forming the working port 202 further comprises making an incision having a length of 6 to 10 cm. The incision length is determined to provide sufficient access while maintaining the benefits of a minimally invasive approach. For present purposes, the dimensions of the incision are optimized to balance surgical access requirements with cosmetic outcomes and recovery time. The specified incision length range facilitates proper exposure of the surgical field while minimizing postoperative complications. The specified incision length allows for proper tissue retraction and instrument manipulation during various steps of the procedure.

At step 106, the method 100 includes docking of robotic surgical system (not shown) and inserting (or forming) a port (herein also referred to as a “second port”) for a first robotic arm at a 2^{nd} intercostal space lateral to a mid clavicular line 204 and inserting a port (herein referred to as a “third port”) for a fourth robotic arm at a 6^{th} intercostal space anterior axillary line 206 configured to receive robotic arms therein. Whereas the ports for second and third robotic arms can be inserted directly through the working port 202. Herein, the ports 204, 206 are positioned to establish optimal triangulation with the working port 202 while maintaining proper spacing for robotic arm movement. Such positioning of these ports 204, 206 ensures appropriate instrument reach and range of motion during the various phases of the transplant procedure. The ports 204, 206 are placed to avoid interference between robotic arms while maximizing the surgical workspace and maintaining optimal angles for tissue manipulation and suturing. The ports 204, 206 are designed to maintain stable positioning throughout the procedure while accommodating the dynamic movements required for complex surgical maneuvers. The port placement also accounts for the need to transition between different surgical phases while maintaining stable access and optimal visualization.

The ports 204, 206 are positioned to provide complementary access points that work in conjunction with the working port 202, creating a system of access for the robotic surgical platform. The spacing between the ports 204, 206 is defined to prevent instrument collisions while enabling full range of motion for the robotic arms throughout the procedure. The placement of the ports 204, 206 also considers the curvature of the chest wall and the relationship between external port positions and internal surgical targets. Specifically, the placement of the ports 204, 206 considers the fulcrum effect of the chest wall and its impact on instrument movement and control. Further, the formation of ports 204, 206 involves consideration of thoracic anatomy of the patient and the planned surgical approach. The formation of ports 204, 206 includes tissue dissection and attention to intercostal neurovascular structures. The port formation process incorporates techniques to minimize tissue trauma while ensuring secure positioning of the robotic arm trocars. The final configuration for the ports 204, 206 is defined to enable efficient instrument manipulation and to maintain pneumostasis during the robotic processes of the procedure.

In some embodiments, the method 100 includes performing a computed tomography (CT) scan of the patient to determine an anatomy of the patient. The CT scan provides three-dimensional visualization of anatomical structures within the thoracic cavity of the patient. The CT scan enables measurement of intercostal spaces and identification of anatomical landmarks for port placement. The CT scan data also allows evaluation of spatial relationships between the thoracic wall and underlying cardiac structures, and thus facilitates preoperative planning of optimal access trajectories through the thoracic cavity.

Further, the method 100 includes determining locations of the working port 202, the second port 204, and the third port 206 based on the determined anatomy of the patient via the CT scan. The CT scan data enables mapping of port locations based on measured anatomical distances and tissue thicknesses. The determined port locations maintain required spacing between ports for preventing interference between robotic arms during operation. The port placement determination uses anatomical measurements from the CT scan to achieve optimal angles for instrument approach and target visualization, and establish proper triangulation for accessing the surgical site through the thoracic cavity.

At step 108, the method 100 includes arranging instruments at the robotic arms. DeBakey forceps can be used at arm no. 1 (the first robotic arm) via the port 204, a 30-degree camera at arm no. 2 (the second robotic arm), Cadiere forceps at arm no. 3 (the third robotic arm) via the port 202 and scissors/needle holders (alternatively) can be used at arm no.4 (the fourth robotic arm) via the port 206.

The DeBakey forceps at the first arm (not shown) helps atraumatic tissue handling, imaging device of the second robotic arm (not shown) provides continuous visualization of the surgical field throughout the procedure. The Cadiere forceps attached to the third robotic arm (not shown) enables additional tissue manipulation capabilities through the working port 202. The concurrent arrangement of the second and third robotic arms through the working port 202 maximizes utilization of the available access space while maintaining proper separation between instruments.

The fourth robotic arm through the port 206 disposed at a 6th support intercostal space anterior axillary line, the fourth robotic arm including scissors or a needle holder. The said position of the fourth robotic arm (not shown) enables cutting and suturing movements during the transplant procedure. The location of the fourth robotic arm relative to the anterior axillary line establishes proper triangulation with other robotic arms for accessing all required surgical targets. The interchangeable scissors and needle holder configuration of the fourth robotic arm provides necessary instrumentation for different phases of the procedure.

The arrangement of the said robotic arms establishes a coordinated system for performing the complex surgical maneuvers required during the transplant procedure. Further, the defined geometric relationships between the robotic arms maintain proper spacing and angles for instrument movement while preventing interference between arms. The present configuration enables smooth transitions between different surgical steps while maintaining stable access to the required operative region.

At step 110, the method 100 includes initiating cardiopulmonary bypass upon achieving a predetermined activated clotting time (ACT), opening a pericardium anteriorly and retracting the pericardium laterally, and dissecting around the SVC, an inferior vena cava (IVC), an aorta, and a pulmonary artery. The initiation of cardiopulmonary bypass occurs only after confirmation of adequate anticoagulation through ACT measurement. The pericardial opening provides direct access to the cardiac structures and great vessels. The anterior pericardial incision enables exposure of the surgical field while maintaining pericardial integrity for later closure. In general, the lateral retraction of the pericardial edges establishes a stable surgical region for subsequent dissection.

In some embodiments, the method 100 includes systematic dissection around each major vessel to prepare for subsequent steps of the transplant procedure. The dissection sequence follows a structured approach to isolate each vascular structure. The SVC dissection includes creation of a passage around the vessel for subsequent snare placement. The IVC dissection extends from the right atrial junction to the diaphragmatic reflection. The aortic dissection encompasses the ascending portion to establish a site for cross-clamping. The pulmonary artery dissection provides access for the subsequent anastomotic phase. Each vessel dissection incorporates careful preservation of surrounding structures while establishing adequate circumferential access for the transplant procedure.

For present purposes, the ACT is 480 s or greater. The specified ACT threshold of 480 seconds ensures adequate anticoagulation for cardiopulmonary bypass. The threshold value prevents thrombus formation within the bypass circuit. It may be understood that the ACT measurement follows standardized testing protocols. Further, the method 100 may implement repeated ACT measurements to maintain required anticoagulation levels.

In some embodiments, the initiating cardiopulmonary bypass includes administering heparin. The heparin administration precedes the initiation of cardiopulmonary bypass to achieve required anticoagulation levels. The present method 100 involves systematic monitoring of coagulation parameters following heparin administration. The anticoagulation protocol ensures safe establishment of the cardiopulmonary bypass circuit. It may be understood that the heparin dosage follows standardized protocols based on patient parameters.

At step 112, the method 100 includes cross-clamping a distal ascending aorta with a cross-clamp, snaring the SVC and IVC, excising a native heart, and removing the native heart via the working port 202. The cross-clamp procedure establishes a bloodless field for the subsequent cardiac excision. The cross-clamping procedure involves precise placement of the cross-clamp on the ascending aorta at a predetermined location. The placement of the cross-clamp maintains adequate distance from the planned aortic anastomosis site. The cross-clamping process may be implemented with careful manipulation to avoid injury to adjacent structures.

Further, the snaring procedure includes sequential occlusion of the major venous channels. The SVC snare placement occurs after confirming proper position of the percutaneous jugular drainage cannula. The IVC snare application follows verification of adequate venous drainage through the femoral cannula. The snaring technique incorporates techniques to prevent tissue damage while ensuring complete vessel occlusion. Such coordinated snaring sequence maintains stable cardiopulmonary bypass flow.

Furthermore, the native heart excision follows a structured approach to separate the heart from its vascular connections. The native heart excision sequence includes systematic division of the great vessels at predetermined locations. The present method 100 involves maintaining adequate tissue cuffs on all vessels for subsequent anastomoses. The process includes systematic inspection of all divided structures to ensure adequate tissue quality for anastomoses. Herein, the native heart excision process may be implemented careful preservation of the posterior pericardial reflection.

The removal of the native heart through the working port 202 can include various maneuvers and manipulations to facilitate extraction. Herein, the working port 202 enable safe passage of the excised heart without requiring extension of the incision. The cardiac manipulation maintains awareness of potential injury to surrounding structures. The extraction process incorporates techniques to protect the thoracic wall during removal. For this purpose, the method 100 includes confirmation of complete hemostasis following heart removal.

At step 114, the method 100 includes arranging, through the working port 202, a donor heart in the patient anatomically. The donor heart positioning incorporates steps to ensure optimal exposure for each anastomosis. The donor heart insertion through the working port 202 can follow a predetermined orientation to ensure proper anatomical alignment. The anatomical arrangement accounts for the sequence of planned anastomoses. The positioning maintains adequate access to all vessel ends requiring connection. The present method 100 includes specific maneuvers to protect the donor heart during passage through the working port 202. The positioning process maintains awareness of the geometric relationships between cardiac structures and their target connections. The arrangement establishes proper orientation for subsequent anastomotic procedures. The present method 100 may also involve verification of proper geometric relationships between all structures before initiating anastomoses. The arrangement process incorporates steps to prevent twisting or distortion of the great vessels.

In some embodiments, the method 100 includes applying a sterile gel to the working port 202 before arranging the donor heart through the working port 202. The application of the sterile gel provides additional lubrication for smooth passage of the donor heart. The gel coating reduces friction between the donor heart and edges of the working port 202. The application of the gel facilitates insertion while protecting the donor heart tissue. The application process maintains sterile technique throughout the procedure.

At step 116, the method 100 includes performing anastomosis of a left atrium using a first suture. The left atrial anastomosis follows a systematic approach to connect donor and recipient tissues. The suturing sequence maintains consistent spacing and depth for each bite. The anastomotic technique incorporates techniques to prevent narrowing or distortion of the atrial connection. Verification of suture line integrity can also be performed before proceeding to subsequent steps.

In an embodiment, the material of the first suture is 3/0 polypropylene. The polypropylene suture material provides sufficient tensile strength for atrial tissue approximation. Further, the material characteristics enable the first suture to have smooth passage through cardiac tissues. The material properties also ensure knot security during the healing phase. It may be appreciated that the suture selection accounts for specific requirements for cardiac tissue handling.

At step 118, the method 100 includes performing anastomosis of the pulmonary artery using a second suture. The pulmonary artery connection incorporates techniques to match vessel sizes between donor and recipient. The anastomotic process maintains proper alignment to prevent vessel kinking. The suturing technique ensures adequate tissue approximation while preventing narrowing. The method 100 may implement systematic inspection of the completed anastomosis for potential issues.

At step 120, the method 100 includes performing anastomosis of the aorta using a third suture and inserting a root vent connected to low suction with cardiopulmonary bypass. The aortic anastomosis follows geometric alignment between donor and recipient vessels. The suturing process maintains consistent technique to ensure uniform connection. The root vent placement provides controlled deairing during subsequent steps. The method 100 may incorporate specific suture placement patterns to prevent distortion or narrowing.

At step 122, the method 100 includes performing anastomosis of the IVC using a fourth suture and removing the aortic cross-clamp. The IVC anastomosis incorporates techniques to match vessel dimensions and prevent narrowing. The suturing process maintains proper tissue orientation throughout the connection. The removal of the aortic cross-clamp follows a controlled sequence to allow gradual reperfusion. The method 100 may include monitoring for adequate hemostasis during this transition.

At step 124, the method 100 includes performing anastomosis of the SVC using a fifth suture on the donor heart. The SVC anastomosis completion occurs with the heart beating to ensure proper alignment under physiological conditions. The suturing technique incorporates techniques to prevent stenosis or distortion. The anastomotic process maintains awareness of adjacent structures during completion. The present method 100 may include final inspection of all connections before proceeding with subsequent steps.

In some embodiments, performing the anastomosis of the IVC and removing the aortic cross-clamp includes removing the aortic cross-clamp while raising an aortic root vent suction to, for example, 400 to 600 cc/min, or 450 to 550 cc/min, or 500 cc/min. The increased suction rate ensures controlled removal of air from the cardiac chambers during reperfusion. The suction adjustment follows a predetermined protocol to prevent air embolism. The process incorporates sequences for gradual restoration of cardiac perfusion. The method 100 may involve systematic monitoring of the root vent output during this phase.

In some embodiments, the method 100 further includes filling the donor heart to check all suture lines, gradually weaning off the cardiopulmonary bypass machine on minimal inotropic support, decannulating the patient, and administering protamine. The cardiac filling process enables systematic inspection of all anastomotic sites under varying pressures. The weaning sequence follows predetermined protocols for establishing adequate cardiac function. The decannulation process incorporates specific steps for maintaining hemodynamic stability. The protamine administration follows standardized protocols for reversal of anticoagulation.

Upon determining a suture line is not formed correctly, such as having a blood leak, the method 100 includes placing additional stitches to correct the suture line. This can help, for example, control bleeding. The inspection process identifies specific areas requiring reinforcement. The additional stitch placement follows the original suturing technique. The correction process maintains proper tissue approximation. The method 100 includes verification of the repair before proceeding with subsequent steps.

In some embodiments, the method 100 also includes performing postoperative transesophageal echocardiography to confirm good left ventricular function and mildly reduced right ventricular function. The method 100 incorporates specific measurements of ventricular function. The echocardiographic assessment evaluates specific parameters of cardiac performance. The imaging sequence includes systematic evaluation of all chambers and valves. Such proposed assessment provides documentation of immediate post-transplant status of cardiac function.

To summarize, the method 100 for performing the robotic-assisted orthotopic heart transplant includes initial preparation steps comprising positioning of the patient in a left lateral decubitus position and establishing airway access using a double lumen endotracheal tube 200. The method 100 proceeds with insertion of monitoring equipment through the left internal jugular vein, including placement of the central venous line and the Swan Gans catheter for continuous hemodynamic monitoring. The surgical access is established through creation of multiple ports, including the working port 202 formed by the mini-thoracotomy, the second port 204 positioned lateral to the mid-clavicular line, and the third port 206 located anterior axillary line.

Further, the method 100 incorporates systematic arrangement of multiple robotic arms through the established ports. The first robotic arm equipped with the first forceps extends through the second port 204, while the second robotic arm containing the imaging device and the third robotic arm with the second forceps operate through the working port 202. The fourth robotic arm containing interchangeable scissors and needle holders functions through the third port 206. The method 100, then, involves bypass phase which includes cannulation of the right groin for cardiopulmonary bypass using the arterial cannula and the multistage femoral venous cannula, accompanied by percutaneous cannulation of the right internal jugular vein for SVC drainage.

The method 100 proceeds with systematic removal of the native heart and implantation of the donor heart through the working port 202. The anastomotic phase follows a structured sequence, beginning with left atrial connection using suture, followed by pulmonary artery anastomosis, aortic anastomosis with root vent placement, IVC anastomosis with concurrent aortic cross-clamp removal, and completion of SVC anastomosis on the heart. The method 100 concludes with weaning from cardiopulmonary bypass, verification of anastomotic integrity, and performance of post-procedure transesophageal echocardiography to confirm ventricular function. The proposed standardized approach of the present method 100 enables completion of a complete orthotopic heart transplant through a minimally invasive robotic-assisted technique while maintaining procedural safety and efficacy.

### EXAMPLES

In an experimental procedure, the present method for performing a robotic-assisted orthotopic heart transplant was implemented in a case of a 16-year-old patient suffering from stage D heart failure due to dilated cardiomyopathy. The patient had a history of cardioembolic stroke seven months prior with no residual weakness, necessitating warfarin therapy. The patient's height was 166 cm, weight 72 kg, BMI 26.1 & BSA 1.8, with blood group A positive. Due to refractory heart failure, the patient was admitted and placed on IV inotropic support. Laboratory data was insignificant except for High INR. The patient had no history of prior cardiac surgery. Transthoracic Echocardiography showed dilated left ventricle with reduced ejection fraction (EF 25%), Dilated right ventricle with moderate to severe dysfunction, moderate to severe tricuspid regurgitation. Right heart catheterization showed mean RA pressure 14 mm Hg, RV systolic pressure 40 mm Hg, mean PA pressure 24 mm Hg, PCW 14 mmHg. PVR was 3 woods unit and Cardiac index was 2.05 L/min/m.

The patient was listed for heart transplant after discussion with a multidisciplinary team. Following identification of a suitable donor, informed consent was obtained for the robotic assisted heart transplant procedure. Warfarin was discontinued, and preoperative neurological clearance was obtained.

A robotic CT (Chest, Abdomen, Pelvis) protocol was performed which demonstrated suitable intrathoracic anatomy and good peripheral vasculature for peripheral cannulation. During hospitalization, a suitable matching donor was identified, and the patient was taken to the operating room.

The procedure was initiated under general anesthesia with double lumen endotracheal tube 200. A central venous line and Swan Gans catheter was inserted through left internal jugular vein. The patient was positioned for cardiopulmonary bypass cannulation (CPB) according to robotic surgical procedure protocols. The patient was placed in slight left lateral decubitus position, and surface marking was performed. Following preparation and draping, the working port 202 was created as a 6-7 cm incision, through a subareolar right mini thoracotomy in the 4th intercostal space.

Three additional 8 mm ports were created for robotic arms and cross clamp. The port for robotic arm No. 1 was inserted through 2nd intercostal space lateral to mid clavicular line for Debakey forceps operation. Arms No. 2 and 3 were inserted directly in working port 202 for camera and Cadiere forceps operation, respectively. The port for arm No. 4 was inserted through 6th intercostal space in anterior axillary line for alternating use of monopolar curved scissor and needle holder. A Chitwood clamp was inserted through 2nd intercostal space anterior axillary line.

Under Echocardiography guidance, the patient was cannulated in right groin for cardiopulmonary bypass with an open groin technique. A 17Fr arterial cannula and 25Fr multistage femoral venous cannulas were used, and another 19 Fr cannula was inserted percutaneously into right internal jugular vein for Superior Vena Cava (SVC) drainage.

The da Vinci Xi robot was docked from a left side of the patient, and targeting was performed. All ports were organized as described. Upon arrival of the donor heart at the hospital premises, Heparin was administered. With ACTs of 480 s, cardiopulmonary bypass was initiated, and the pericardium was opened anteriorly and retracted laterally. Careful dissection was performed around SVC, Inferior Vena Cava (IVC), Aorta and pulmonary artery. Following arrival of the donor heart, the distal ascending aorta was cross clamped with Chitwood clamp, and SVC and IVC were snared. The native heart was excised and removed via working port 202. The left atrial cuff, pulmonary artery, aorta, SVC & IVC were reinspected for further trimming and prepared for anastomosis.

A sterile gel was applied over the working port 202, enabling insertion of the donor heart from apex to base without resistance. The heart was positioned anatomically, and left atrial anastomosis was performed using 3/0 polypropylene. The suture was secured using a knot pusher. The pulmonary artery edges were trimmed to match size, and end-to-end anastomosis was performed using CV-4 Gore-Tex suture. Subsequently, end-to-end aortic anastomosis was performed utilizing CV-4 Gore-Tex suture. Following aortic anastomosis, a root vent was inserted and connected to low suction with cardiopulmonary bypass. End-to-end IVC anastomosis was then performed using CV-4 Gore-Tex suture. During removal of the aortic cross-clamp, the Aortic root vent suction was increased to 500 cc/min. The final end-to-end anastomosis of SVC was performed using 5/0 polypropylene suture on the beating heart.

The heart was allowed sufficient perfusion time, during which the heart was gradually filled to check all suture lines, with additional stitches placed where required. The cardiopulmonary bypass machine was gradually weaned off with minimal inotropic support. The patient was decannulated and protamine was administered. The procedure achieved a cold ischemia time of 70 min, warm ischemia time of 143 min, and total ischemia time of 213 min. Reperfusion time was 60 min and total cardiopulmonary bypass time was 250 min.

Post-operative transesophageal echocardiography demonstrated good left ventricular function with EF 55%, mildly reduced right ventricular function. The mean pulmonary artery pressure was measured at 24 mmHg. The patient was transferred to CS-ICU with stable hemodynamics.

The post-operative course proceeded as follows: The patient was extubated on postoperative day (POD) 1, discharged from ICU and hospital on POD 4 and 14, respectively. His post-transplant right heart catheterization revealed a mean right atrial pressure of 9 mmHg, a mean pulmonary artery (PA) pressure of 24 mmHg, and a pulmonary capillary wedge of 12 mmHg. His endomyocardial biopsy result indicated no acute allograft rejection (ISHLT Grade 0R).

The described method was successfully implemented in a patient with stage D heart failure, demonstrating the feasibility of completing a full cardiac transplantation through minimally invasive robotic access. The procedure achieved total ischemia time of 213 minutes. Post-operative outcomes showed marked improvement in cardiac function with ejection fraction at 55%, and the patient demonstrated stable hemodynamics with rapid recovery, achieving mobilization by post-operative day three. The systematic port placement and robotic arm arrangement enabled completion of all surgical steps through minimal access points while maintaining surgical precision, suggesting the potential for applying this method to reduce surgical trauma in cardiac transplantation procedures.

The method 100 of the present disclosure, as designed to be implemented for performing a robotic-assisted orthotopic heart transplant, integrates multiple technological components into a standardized surgical approach for complete cardiac transplantation through minimal access ports. The method 100 establishes specific port placements and robotic arm arrangements that enable performance of complex cardiac procedures through defined working spaces. The systematic sequencing of surgical steps, from initial preparation through final anastomosis, creates a reproducible framework for minimally invasive cardiac transplantation. The method 100 incorporates precise positioning of robotic instruments and specific approaches to vessel management that maintain surgical accuracy while reducing access requirements. The integration of imaging guidance, standardized port placement, and structured surgical sequences enables completion of all required steps through the working port and supporting robotic access points. The systematic arrangement of surgical steps enables completion of complex anastomoses through precisely positioned robotic instruments.

The method 100 reduces requirements for extensive thoracic access while maintaining complete surgical control through standardized port placement and robotic assistance. The establishment of specific geometric relationships between multiple robotic arms enables coordinated instrument movements through minimal access points. The method 100 maintains continuous visualization and instrument access throughout the procedure through defined port arrangements. The incorporation of peripheral cannulation techniques eliminates requirements for direct central vessel access. The structured approach to donor heart insertion and implantation through the working port reduces requirements for extensive tissue dissection.

Numerous modifications and variations of the present disclosure are possible in light of the above teachings. It is therefore to be understood that within the scope of the appended claims, the invention may be practiced otherwise than as specifically described herein.

1. A method for performing a robotic-assisted orthotopic heart transplant, comprising:
forming a first port subareolarly by a mini-thoracotomy at a working intercostal space of a patient, the first port being a working port;
forming a second port and a third port configured to receive robotic arms therein;
arranging a first robotic arm through the second port disposed at a second intercostal space lateral to a mid-clavicular line, the first robotic arm including first forceps;
arranging a second robotic arm and a third robotic arm in the working port, the second robotic arm including an imaging device and the third robotic arm including second forceps;
arranging a fourth robotic arm through the third port disposed at a fourth intercostal space proximal to an anterior axillary line, the fourth robotic arm including scissors or a needle holder;
cannulating the patient in a right groin for cardiopulmonary bypass with an arterial cannula and a multistage femoral venous cannula, and inserting a cannula percutaneously into a right internal jugular vein for superior vena cava (SVC) drainage;
initiating cardiopulmonary bypass upon achieving a predetermined activated clotting time (ACT), opening a pericardium anteriorly and retracting the pericardium laterally, and dissecting around the SVC, an inferior vena cava (IVC), an aorta, and a pulmonary artery;
cross-clamping a distal ascending aorta with a cross-clamp, snaring the SVC and IVC, excising a native heart, and removing the native heart via the working port;
arranging, through the working port, a donor heart in the patient anatomically;
performing anastomosis of a left atrium using a first suture;
performing anastomosis of the pulmonary artery using a second suture;
performing anastomosis of the aorta using a second suture and inserting a root vent connected to low suction with cardiopulmonary bypass;
performing anastomosis of the IVC using a third suture and removing the aortic cross-clamp; and
performing anastomosis of the SVC using a fourth suture on the donor heart.

2. The method of claim 1, further comprising arranging a patient in a left lateral decubitus position under general anesthesia with a double lumen endotracheal tube.

3. The method of claim 1, further comprising inserting a central venous line and a pulmonary artery catheter through a left internal jugular vein.

4. The method of claim 3, wherein the pulmonary artery catheter is a Swan Gans catheter.

5. The method of claim 1, further comprising arranging a multiport robotic arm platform proximal to a left side of the patient, the multiport robotic arm platform configured to control the robotic arms.

6. The method of claim 1, further comprising filling the donor heart to check all suture lines, gradually weaning off the cardiopulmonary bypass machine on minimal inotropic support, decannulating the patient, and administering protamine.

7. The method of claim 6, further comprising upon determining a suture line is not formed correctly, placing additional stitches to correct the suture line.

8. The method of claim 1, further comprising performing postoperative transesophageal echocardiography to confirm good left ventricular function and mildly reduced right ventricular function.

9. The method of claim 1, wherein the working port has a diameter of 5 to 8 cm.

10. The method of claim 1, wherein forming the working port further comprises making an incision having a length of 6 to 10 cm.

11. The method of claim 1, wherein a diameter of the second port is 7 to 9 mm.

12. The method of claim 1, further comprising applying a sterile gel to the working port before arranging the donor heart through the working port.

13. The method of claim 1, wherein the initiating pulmonary bypass further comprises administering heparin.

14. The method of claim 1, wherein the performing the anastomosis of the IVC and removing the aortic cross-clamp further comprises removing the aortic cross-clamp while raising an aortic root vent suction to 500 cc/min.

15. The method of claim 1, wherein the cannulating the patient in the right groin for cardiopulmonary bypass further comprises cannulating the patient in the right groin for cardiopulmonary bypass under echocardiography guidance.

16. The method of claim 1, wherein the cannulating the patient in the right groin for cardiopulmonary bypass uses an open groin technique.

17. The method of claim 1, further comprising performing a computed tomography (CT) scan of the patient to determine an anatomy of the patient.

18. The method of claim 17, further comprising determining locations of the working port, the second port, and the third port based on the determined anatomy of the patient via the CT scan.

19. The method of claim 1, wherein the ACT is 480 s or greater.

20. The method of claim 1, wherein a material of the first suture is 3/0 polypropylene;
wherein a material of the second, third, and fourth suture is CV-4 polytetrafluoroethylene; and
wherein a material of the fifth suture is 5/0 polypropylene.
